(19) United States
(12) Patent Application Publication
(10) Pub. No.: US 2026/0354809 A1
(43) Pub. Date: Nov. 12, 2026

(54) MEDICAL DEVICE

(71) Applicant: TERUMO KABUSHIKI KAISHA, TOKYO (JP)

(72) Inventor: Yusaku YAMAKI, kanagawa (JP)

(21) Appl. No.: 19/714,828

(22) Filed: Jun. 22, 2026

Related U.S. Application Data
(63) Continuation of application No. PCT/JP2024/045475, filed on Dec. 23, 2024.

(30) Foreign Application Priority Data
Dec. 25, 2023 (JP) 2023217650

(51) Int. Cl. A61B 17/02; A61B 17/00

(52) U.S. Cl. CPC A61B 17/02; A61B 2017/00367; A61B 2017/00477; A61B 2017/00566; A61B 2017/0243

(57) ABSTRACT

A medical device (10) suppresses movement of a surface of a heart (300) by being brought into contact with the heart (300) of a living body (P). The medical device (10) includes openable and closable first and second arm portions (141, 142) that include a pair of suction portions (58) for suctioning the heart (300) and are supported by a shaft (12). The first and second arm portions (141, 142) constitute a distal portion of the medical device (10).

### CROSS REFERENCE TO RELATED APPLICATIONS

This application is a continuation of PCT Application No. PCT/JP2024/045475, filed on Dec. 23, 2024, based on and claiming priority to Japanese Application No. JP2023217650, filed on Dec. 25, 2023, both of which are incorporated by reference in their entirety.

### BACKGROUND OF THE INVENTION

The present invention relates to a medical device.

JP 2023-73318 A discloses a medical device for controlling beating of a heart of a human body. The medical device includes a pair of arms attached to a rail of a sternal retractor, and a pair of contact portions provided at respective distal ends of the pair of arms. In open-chest surgery, the pair of contact portions is inserted into a living body through an opening, and ends of the pair of contact portions are brought into contact with a surface of the heart, so that movement of the surface caused by beating of the heart is controlled.

In open-chest surgery, in order to reduce a burden on a living body, a small opening is formed near an incision, a shaft is inserted into the living body through the opening, and a medical device is attached to a distal end of the shaft. In this case, after the shaft is inserted into the living body from the opening in a state in which a cap is attached to the distal end of the shaft, the cap needs to be removed inside the living body, and a medical device (stabilizer) needs to be attached to the distal end of the shaft.

However, removal of the cap inside the living body and an operation of attaching the medical device to the distal end of the shaft are complicated, and furthermore, after the cap is removed inside the living body, there is a concern that the cap may be accidentally dropped inside the living body (surgical field). In this case, an additional operation that would not originally occur, namely collecting the dropped cap from inside the living body (surgical field), must be performed.

### SUMMARY OF THE INVENTION

An object of the present invention is to solve the above-described problem.

An aspect of the present invention is a medical device that suppresses movement of a surface of an organ of a living body by being brought into contact with the organ, the medical device including: a shaft that is inserted into the living body; and a pair of arm portions that constitute a distal portion of the medical device, include a pair of suction portions that suction the organ, and are supported by the shaft, the pair of arm portions being openable and closable.

According to this medical device, since the pair of arm portions is openable and closable, the pair of arm portions is capable of being inserted into the living body through an opening provided in the living body in a state in which the pair of arm portions is closed. Therefore, unlike a structure in which a cap is removed from a distal end of a shaft inside a living body and a medical device (stabilizer) is attached to the distal end of the shaft, occurrence of an event in which the cap is dropped into a surgical field is capable of being avoided.

In the medical device according to the first aspect of the present invention, the shaft may include an outer rod having a tubular shape; an inner rod inserted through a lumen of the outer rod; and a support structure provided at a distal end of the inner rod and supporting the pair of arm portions in an openable and closable manner, the outer rod and the inner rod may be relatively movable in an axial direction of the outer rod, and the outer rod may include an arm accommodation space capable of accommodating at least proximal portions of the pair of arm portions in a state in which the pair of arm portions is closed.

With this configuration, since the pair of arm portions is accommodated in the arm accommodation space of the outer rod in a state in which the pair of arm portions is closed, insertion resistance of the pair of arm portions is effectively suppressed when the pair of arm portions is inserted into the living body.

In the medical device according to above aspect of the present invention, at a first position where the outer rod has moved in a distal direction relative to the inner rod, the arm portions may enter a closed state in which distal ends of the pair of arm portions have rotated in a direction approaching each other about a support portion of the support structure as a fulcrum, and at a second position where the outer rod has moved in a proximal direction relative to the inner rod, the arm portions may enter an open state in which the distal ends of the pair of arm portions have rotated so as to be separated from each other about the support portion of the support structure as a fulcrum.

With this configuration, the arm portions are inserted into the living body at the first position where the arm portions are in the closed state, and the arm portions are rotated inside the living body to be set to the opened second position, so that both insertability of the arm portions into the living body and retainability of a surface of an organ are capable of being achieved.

The medical device according to above aspects of the present invention may include: an operation portion that is provided at a proximal portion of the outer rod and performs opening and closing operations of the pair of arm portions.

With this configuration, by operating the operation portion, opening and closing operations of the pair of arm portions are capable of being easily performed.

In the medical device according to above aspects of the present invention, the operation portion may move the outer rod in the axial direction relative to the inner rod.

With this configuration, by operating the operation portion, the outer rod is capable of being easily moved in the axial direction relative to the inner rod.

In the medical device according to above aspects of the present invention, in the open state of the arm portions, when the outer rod is moved from the second position in the distal direction relative to the inner rod, the arm portions may enter the closed state in which the distal ends of the pair of arm portions have rotated in a direction approaching each other about the support portion of the support structure as a fulcrum.

With this configuration, when the arm portions are removed from inside the living body after a procedure, the arm portions are capable of being closed and taken out in a compact state.

In the medical device according to above aspects of the present invention, the outer rod may include a slit portion that opens in a distal end surface of the outer rod and extends from the distal end surface in the proximal direction, in a state in which at least the proximal portions of the pair of arm portions are accommodated in the arm accommodation space of the outer rod, a protrusion provided on the support structure may be inserted into the slit portion, and in a state in which the arm portions are placed in the open state by moving the outer rod in the proximal direction relative to the inner rod, the protrusion of the support structure may be disengaged from the slit portion.

With this configuration, when the outer rod and the inner rod are relatively moved in the axial direction by operation of the operation portion, the support structure is capable of being guided in the axial direction of the outer rod by the slit portion.

In the medical device according to above aspects of the present invention, the support structure may include a first support member provided at a distal portion of the inner rod, a second support member provided so as to be relatively movable in the axial direction with respect to the first support member, and a protrusion provided on an outer wall of the second support member and protruding radially outward, and after the entirety of the pair of arm portions protrudes from a distal opening of the outer rod and the pair of arm portions enters the open state, when the outer rod is moved in the distal direction relative to the inner rod in a state in which the protrusion and the distal end surface of the outer rod face each other in the axial direction, the protrusion may abut against a distal end of the outer rod, and the second support member may move relative to the first support member in the distal direction, so that the pair of arm portions enters the closed state.

With this configuration, after the pair of arm portions is made to protrude in the distal direction from the distal end of the outer rod, the distal end of the outer rod is pressed against the protrusion, so that the second support member is capable of being moved in a direction away from the first support member to close the arm portions.

In the medical device according to above aspects of the present invention, the support structure may include: a first support member provided at a distal portion of the inner rod, a second support member provided so as to be relatively movable in the axial direction with respect to the first support member, a protrusion provided on an outer wall of the second support member and protruding radially outward, and an arm lock portion that releasably restricts opening and closing operations of the arm portions in an open state of the pair of arm portions, the arm lock portion may include a first attraction member provided on the first support member, and a second attraction member provided on the second support member and attractable to the first attraction member, and in the open state of the pair of arm portions, the first attraction member and the second attraction member may be attracted to each other, and relative movement of the first support member and the second support member in the axial direction may be restricted.

With this configuration, the open state of the pair of arm portions is effectively maintained by the arm lock portion.

In the medical device according to above aspects of the present invention, the outer rod may include a slit portion that opens in a distal end surface of the outer rod and extends from the distal end surface in a proximal direction, at a first position that is a closed state of the pair of arm portions, the protrusion may be inserted through the slit portion of the outer rod, at a second position that is the open state of the arm portions, the protrusion may be disengaged from the slit portion and disposed on a distal side with respect to the distal end surface of the outer rod, and when the outer rod moves in a distal direction relative to the inner rod from the second position in a state in which the protrusion and the distal end surface of the outer rod face each other in the axial direction, the protrusion may abut against the distal end surface of the outer rod and push the protrusion in the distal direction, so that an attraction state between the first attraction member and the second attraction member is released.

With this configuration, when the arm portions are accommodated in the outer rod after surgery, the distal end of the outer rod is brought into contact with the protrusion, so that the open state of the arm portions by the arm lock portion is capable of being released and the arm portions are capable of being closed. By recognizing that the protrusion has abutted against the distal end of the outer rod, a user is capable of effectively confirming that the arm portions have entered a state of being accommodated in the arm accommodation space of the outer rod.

In the medical device according to above aspects of the present invention, at least either one of the first attraction member or the second attraction member may be a magnet.

With this configuration, by forming at least either one of the first attraction member or the second attraction member from a magnet, relative movement of the first support member and the second support member is capable of being effectively restricted.

The medical device according to above aspects of the present invention may include: a movable mechanism that is provided at a distal portion of the inner rod and tiltably supports the support structure with respect to the distal portion of the inner rod, and the movable mechanism may be a joint structure including an engaging projection having a spherical shape provided on either one of the support structure or the inner rod, and an engaging recess provided on the other of the support structure or the inner rod and with which the engaging projection is rotatably engaged.

With this configuration, by tilting the support structure at the distal portion of the inner rod by the movable mechanism, the pair of arm portions is capable of being effectively brought into contact with a desired contact site on an organ.

The medical device according to above aspects of the present invention may include a rotation lock portion that restricts a rotation operation of the support structure with respect to the inner rod by the movable mechanism, in which the rotation lock portion may include a lock member that prevents rotation of the engaging projection with respect to the engaging recess by biasing the engaging recess and the engaging projection in directions approaching each other.

With this configuration, in a state in which the pair of arm portions is tilted with respect to the distal portion of the inner rod by the movable mechanism and brought into contact with a contact site of an organ, a rotation operation of the support structure is locked by the lock member of the rotation lock portion, so that a contact state of the arm portions with respect to the contact site of the organ is effectively maintained.

In the medical device according to above aspects of the present invention, a distal end of each of the arm portions may include a curved portion curved so as to be convex toward the distal end.

With this configuration, when the arm portions are inserted into the living body, insertion resistance and a burden on the living body are capable of being reduced.

According to the present invention, in a medical device that suppresses movement of a surface of an organ of a living body by being brought into contact with the organ, by including a pair of openable and closable arm portions that include a pair of suction portions that suction the organ and are supported by a shaft, the pair of arm portions is capable of being inserted into the living body through an opening provided in the living body in a state in which the pair of arm portions is closed. Therefore, unlike a structure in which a cap is removed from a distal end of a shaft inside a living body and a stabilizer is attached to the distal end of the shaft, occurrence of an event in which the cap is dropped into a surgical field is capable of being avoided.

### BRIEF DESCRIPTION OF THE DRAWINGS

FIG. 1 is an explanatory view showing a use state of a medical device according to an embodiment of the present invention.

FIG. 2 is a side view of the medical device of FIG. 1.

FIG. 3 is an external perspective view showing a distal portion of the medical device.

FIG. 4 is a cross-sectional view taken along line IV-IV in FIG. 2.

FIG. 5 is a side view showing an initial state of the medical device in which first and second arm portions are accommodated.

FIG. 6 is an exploded perspective view of a portion of the distal portion of the medical device shown in FIG. 3.

FIG. 7 is a cross-sectional view taken along line VII-VII in FIG. 2.

FIG. 8 is an explanatory view of a support structure in an open state of the first and second arm portions.

FIG. 9 is a cross-sectional view taken along line IX-IX in FIG. 5.

FIG. 10 is an explanatory view of the support structure in a closed state of the first and second arm portions.

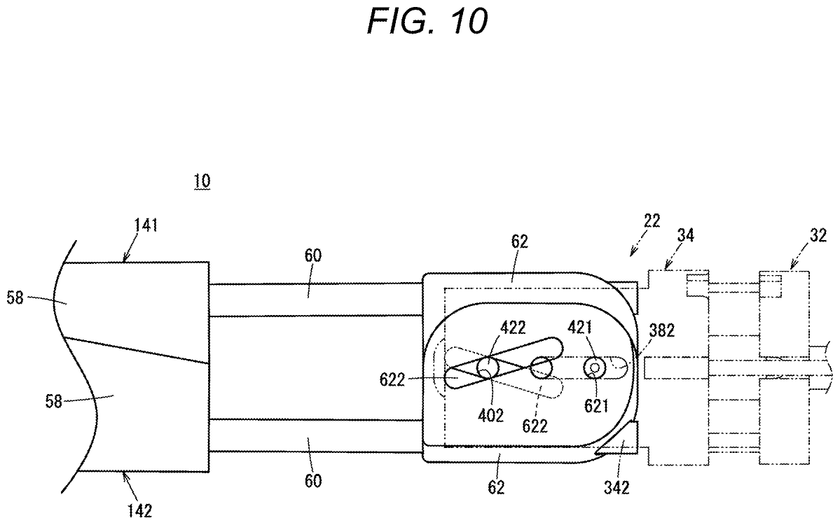

FIG. 11 is a side view showing a state in which an outer rod of the medical device shown in FIG. 5 has been moved in a proximal direction.

FIG. 12 is a perspective view showing a state in which a part of the first and second arm portions is accommodated in the outer rod.

FIG. 13 is a side view showing a distal portion of the medical device of FIG. 12.

### DESCRIPTION OF PREFERRED EMBODIMENTS

Hereinafter, a medical device according to the present invention will be described by giving a preferred embodiment with reference to the accompanying drawings.

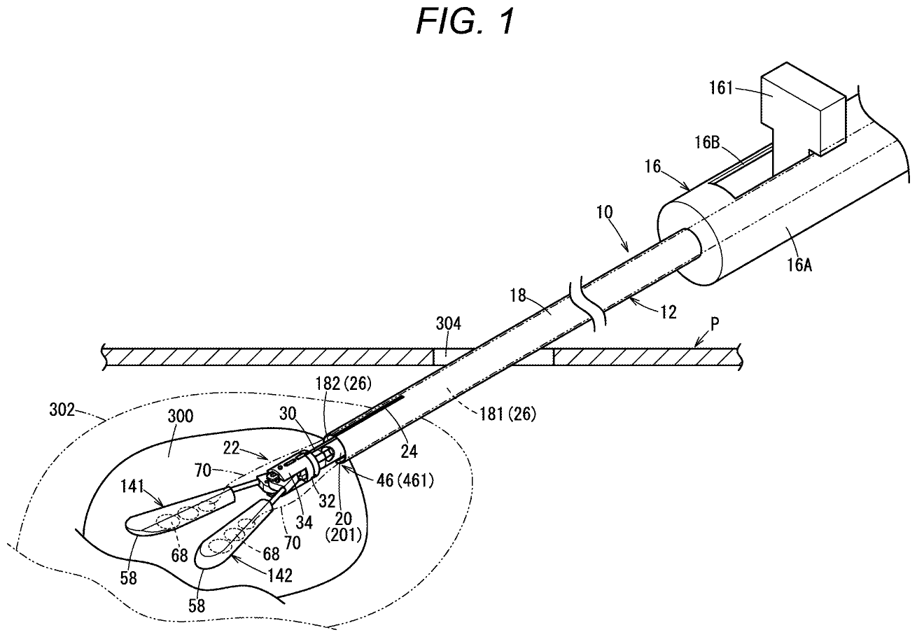

With reference to the drawings, and in particular FIG. 1, an example of a medical device 10 according to the present embodiment is a stabilizer for holding an organ, such as a heart 300, in a desired posture and suppressing movement of a surface of the heart 300 by being brought into contact with an organ (for example, the heart 300) of a human body (hereinafter, a living body P). As shown in FIG. 1, during open-chest surgery (for example, coronary artery bypass surgery), a distal portion of the medical device 10 is inserted into the living body through an opening 304 that is incised adjacent to an incision 302 of the living body P and that is smaller than the incision 302.

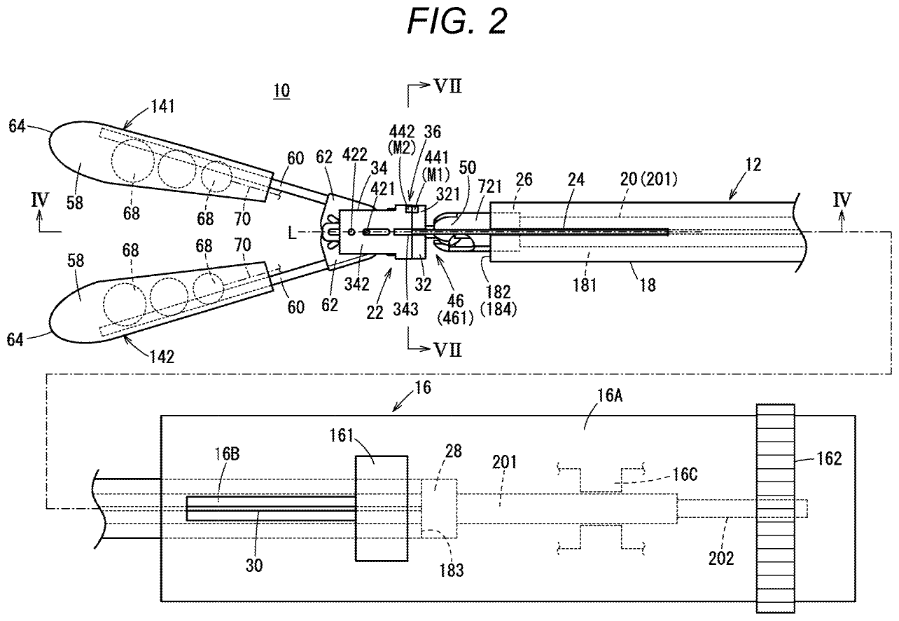

As shown in FIG. 2, the medical device 10 includes a shaft 12, first and second arm portions 141 and 142, and an operation portion 16.

The shaft 12 is inserted into the living body (see FIG. 1). The shaft 12 includes an outer rod 18, an inner rod 20, and a support structure 22 that supports the first and second arm portions 141 and 142 in an openable and closable manner.

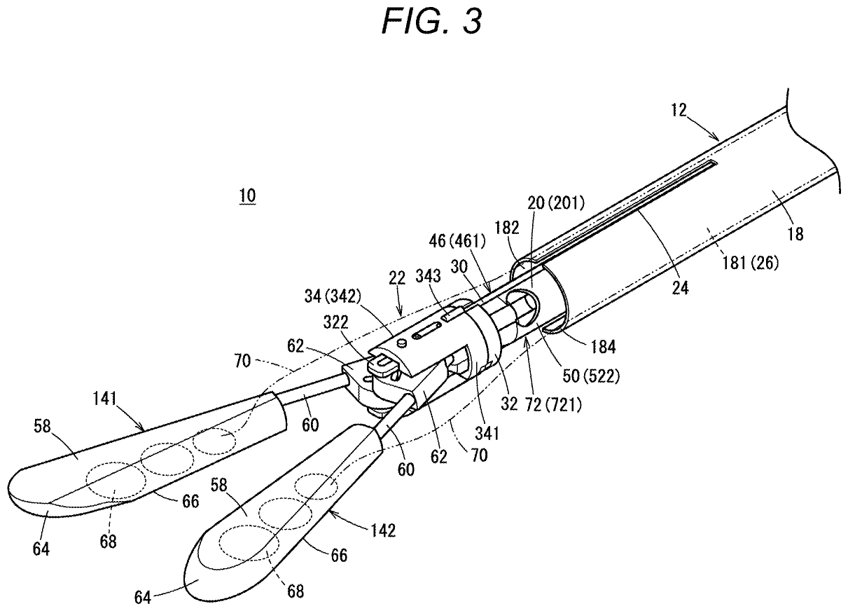

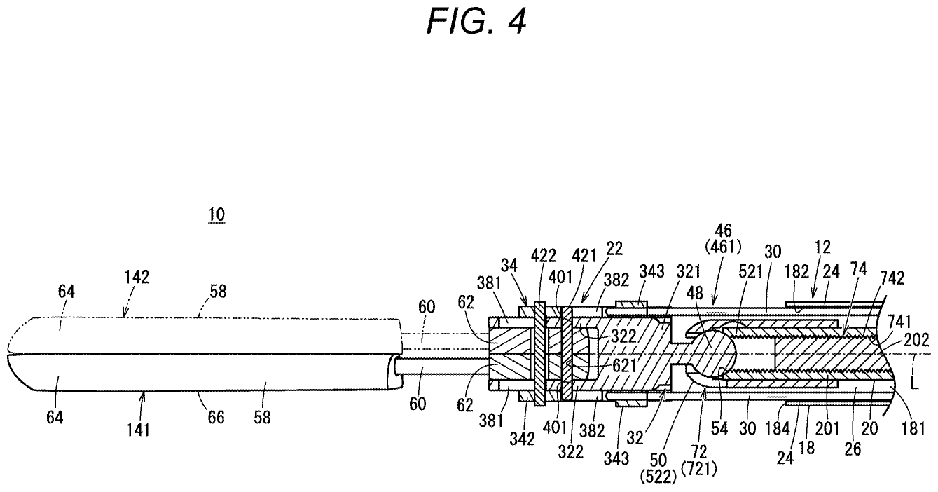

As shown in FIGS. 1-3, the outer rod 18 is formed in a cylindrical shape having a lumen 181 (see FIG. 3). The outer rod 18 includes a distal opening 182, a slit portion 24, and a proximal opening 183. The distal opening 182 opens at a distal end of the outer rod 18. The distal opening 182 and the lumen 181 communicate with each other. The slit portion 24 opens in a distal end surface 184 of the outer rod 18. The slit portion 24 extends from the distal end surface 184 of the outer rod 18 in a proximal direction. As shown in FIG. 4, the slit portion 24 penetrates an outer wall of the outer rod 18 in a thickness direction. A pair of slit portions 24 is provided so as to be separated in a circumferential direction of the outer rod 18. The slit portion 24 is not limited to a case in which a pair of slit portions 24 is provided. One or more slit portions 24 need only be provided.

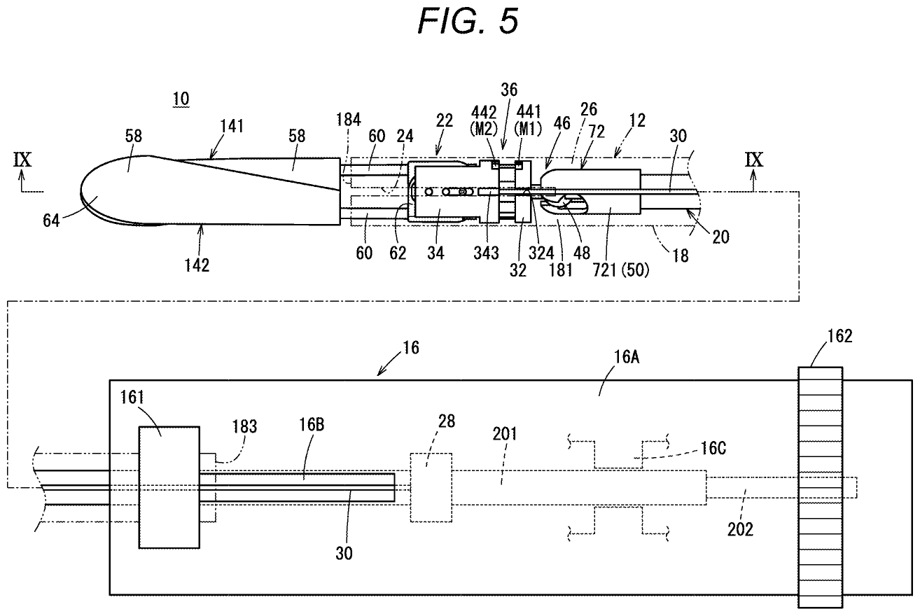

As shown in FIG. 4, a distal portion of the outer rod 18 has an arm accommodation space 26. The arm accommodation space 26 is a part of the lumen 181 at the distal portion of the outer rod 18. As shown in FIG. 5, in a state in which the first and second arm portions 141 and 142 are closed, the arm accommodation space 26 is capable of accommodating at least proximal portions of the first and second arm portions 141 and 142.

As shown in FIG. 4, the inner rod 20 is inserted through the lumen 181 of the outer rod 18. The inner rod 20 and the outer rod 18 are relatively movable in an axial direction of the outer rod 18. The inner rod 20 includes a first rod 201 having a tubular shape and a second rod 202 having a tubular shape. A length of the first rod 201 along the axial direction is longer than a length of the outer rod 18 along the axial direction. A proximal portion of the first rod 201 is disposed in a proximal direction with respect to a proximal portion of the outer rod 18 (see FIG. 2).

As shown in FIG. 2, a collar member 28 is provided on the first rod 201. The collar member 28 is formed in an annular shape covering an outer circumference of the first rod 201. The collar member 28 is provided so as to be movable in the axial direction with respect to the first rod 201. When the inner rod 20 is inserted through the lumen 181 of the outer rod 18, the collar member 28 is disposed in the proximal direction with respect to a proximal end of the outer rod 18. A proximal end of a connecting member 30 is connected to the collar member 28. The connecting member 30 is, for example, a wire member. The collar member 28 and the support structure 22 are connected by the connecting member 30. The connecting member 30 is inserted through the lumen 181 of the outer rod 18.

As shown in FIG. 5, at the time of accommodation in which proximal ends of the first and second arm portions 141 and 142 are accommodated in the arm accommodation space 26, the proximal end of the outer rod 18 and the collar member 28 are disposed so as to be separated in the axial direction.

The second rod 202 is inserted through an inside of the first rod 201. A diameter of the second rod 202 is smaller than a diameter of the first rod 201. A length of the second rod 202 along the axial direction is longer than a length of the first rod 201 along the axial direction. A proximal portion of the second rod 202 is disposed in the proximal direction with respect to the proximal portion of the first rod 201 (see FIG. 2).

As shown in FIG. 2, the support structure 22 is provided at a distal end of the inner rod 20. The support structure 22 supports the first and second arm portions 141 and 142 in an openable and closable manner. The support structure 22 includes a first support member 32, a second support member 34, and an arm lock portion 36. As shown in FIG. 5, when a part of the first and second arm portions 141 and 142 is accommodated in the arm accommodation space 26 of the outer rod 18, the support structure 22 is accommodated in the lumen 181 of the outer rod 18. As shown in FIG. 2, when the first and second arm portions 141 and 142 are disposed in the distal direction from the distal end of the outer rod 18, the support structure 22 is exposed from the distal end of the outer rod 18.

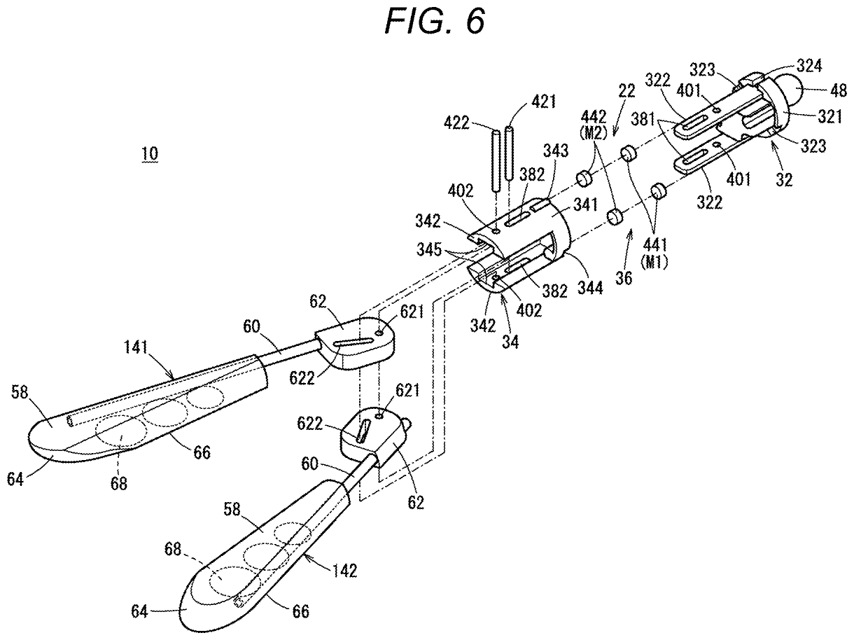

The first support member 32 is provided at a distal portion of the inner rod 20. As shown in FIG. 6, the first support member 32 includes a base portion 321 and a pair of support pieces 322 protruding in the distal direction with respect to the base portion 321. The base portion 321 is provided at a proximal portion of the first support member 32. The base portion 321 has a disk shape orthogonal to an axial direction of the first support member 32. The base portion 321 includes a first accommodation portion 323. The first accommodation portion 323 is a hole that opens in a distal end surface of the base portion 321 facing in the distal direction. A plurality of first accommodation portions 323 are provided so as to be separated in a circumferential direction of the base portion 321.

An outer circumferential surface of the base portion 321 has a groove portion 324 recessed radially inward. The groove portion 324 extends in an axial direction of the base portion 321. A plurality of groove portions 324 are provided so as to be separated in the circumferential direction of the base portion 321. The connecting member 30 is inserted through the groove portions 324 (see FIG. 5).

The pair of support pieces 322 is formed parallel to the axial direction of the first support member 32. The pair of support pieces 322 face each other and are parallel to each other. Each support piece 322 includes a first pin groove 381 and a first pin hole 401. The first pin groove 381 extends from a distal portion of the support piece 322 toward the proximal direction. The first pin hole 401 is disposed so as to be separated in the proximal direction from a proximal end of the first pin groove 381. Each first pin groove 381 and each first pin hole 401 penetrate the support piece 322 in a thickness direction. A first pin member 421 is inserted through each first pin hole 401. The first pin member 421 is supported by the pair of support pieces 322 in a state of being inserted through the first pin holes 401. A second pin member 422 is inserted through each first pin groove 381.

As shown in FIG. 2, the second support member 34 is relatively movable in the axial direction of the inner rod 20 with respect to the first support member 32.

As shown in FIG. 6, the second support member 34 includes an annular portion 341, a pair of overhanging portions 342 extending from the annular portion 341 in the distal direction, and a pair of protrusions 343. The annular portion 341 is provided at a proximal end of the second support member 34. The annular portion 341 has an annular shape centered on an axis of the second support member 34. A distal end of the connecting member 30 is connected to a proximal end of the annular portion 341. The annular portion 341 is disposed in the distal direction with respect to the base portion 321 of the first support member 32.

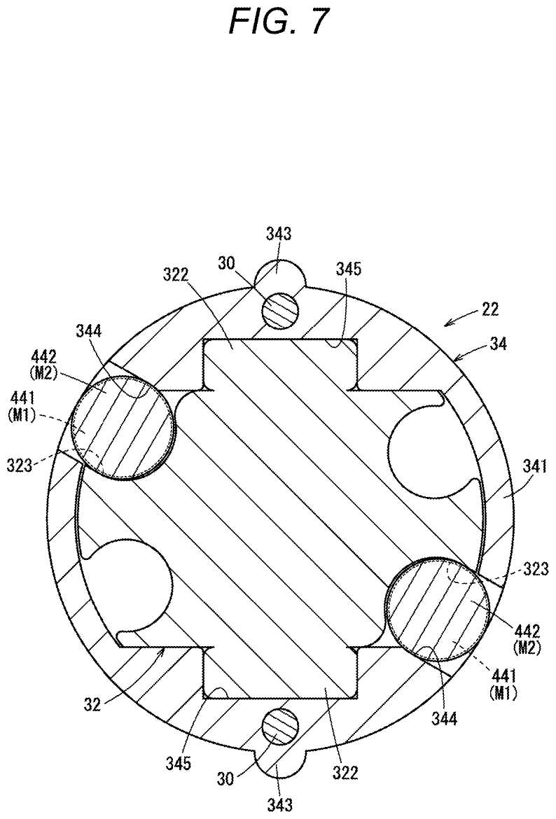

The proximal end of the annular portion 341 includes a second accommodation portion 344. The second accommodation portion 344 is a recessed portion recessed from the proximal end of the annular portion 341 in the distal direction. A plurality of second accommodation portions 344 are provided so as to be separated in the circumferential direction of the annular portion 341. As shown in FIG. 7, in the circumferential direction of the first and second support members 32 and 34, the first accommodation portions 323 and the second accommodation portions 344 face each other. The number of the first accommodation portions 323 and the number of the second accommodation portions 344 are the same.

As shown in FIG. 6, the pair of overhanging portions 342 is formed parallel to the axial direction of the second support member 34. The overhanging portions 342 are provided so as to face each other and be parallel to each other. Each of the overhanging portion 342 includes a second pin hole 402 and a second pin groove 382. The second pin hole 402 is provided in a distal portion of the overhanging portion 342. The second pin groove 382 is provided in the proximal direction with respect to the second pin hole 402. The second pin groove 382 extends along the axial direction of the second support member 34. Each second pin hole 402 and each second pin groove 382 penetrate the overhanging portion 342 in a thickness direction.

An inner surface of each overhanging portion 342 has a guide groove 345 recessed radially outward. The guide groove 345 is provided at a position facing the second pin groove 382 and the second pin hole 402. The support piece 322 of the first support member 32 is inserted into the guide groove 345 (see FIG. 7). When the first support member 32 and the second support member 34 move relatively in the axial direction, the support piece 322 is guided along the guide groove 345.

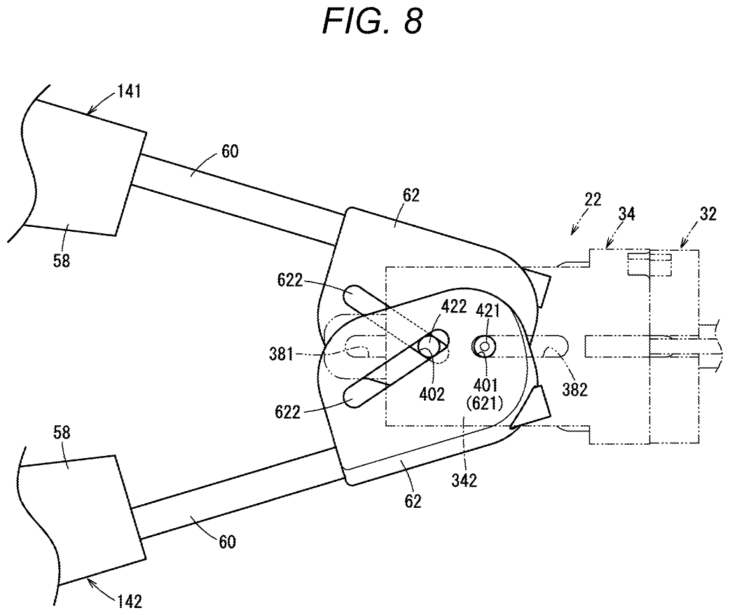

As shown in FIG. 8, in a side view of the medical device 10, the first pin hole 401 and the second pin groove 382 are disposed at the same position, and the first pin groove 381 and the second pin hole 402 are disposed at the same position. End portions of the second pin member 422 inserted through the first pin grooves 381 of the first support member 32 are respectively inserted through the second pin holes 402 of the overhanging portions 342. End portions of the first pin member 421 inserted through the first pin holes 401 of the first support member 32 are respectively inserted through the second pin grooves 382.

As shown in FIG. 6, the pair of protrusions 343 is provided on an outer circumferential surface of the annular portion 341. The protrusions 343 protrude outward from the outer circumferential surface of the annular portion 341. The protrusions 343 extend from the proximal end of the second support member 34 (annular portion 341) in the distal direction. The pair of protrusions 343 is disposed so as to be separated in the circumferential direction of the annular portion 341 (see FIG. 7).

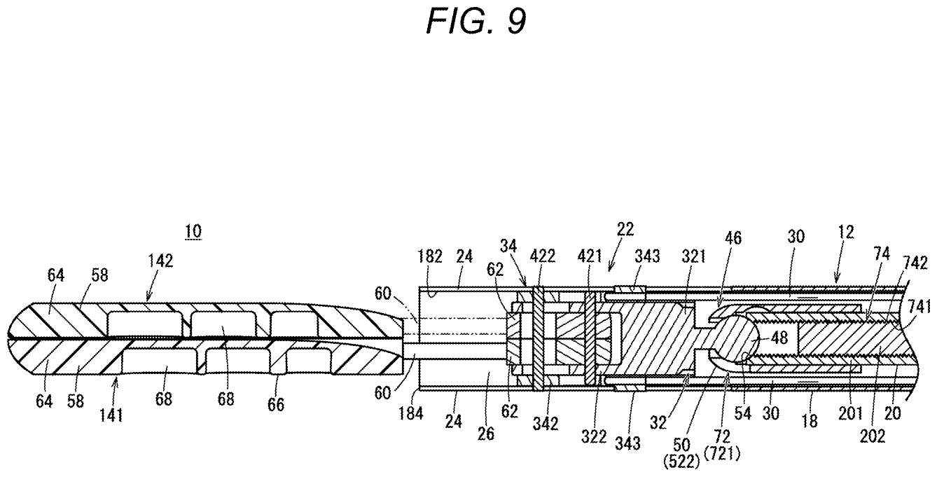

As shown in FIG. 9, in a state in which proximal portions of the first and second arm portions 141 and 142 are accommodated in the arm accommodation space 26 of the outer rod 18, the protrusions 343 provided on the support structure 22 are inserted into the slit portions 24. At the time of accommodation of the first and second arm portions 141 and 142, the protrusions 343 are inserted through the slit portions 24, so that contact between the protrusions 343 and the outer wall (distal end surface 184) of the outer rod 18 is avoided.

As shown in FIG. 2, the arm lock portion 36 releasably restricts opening and closing operations of the first and second arm portions 141 and 142 in an open state in which the first and second arm portions 141 and 142 are open (i.e., separated). As shown in FIG. 6, the arm lock portion 36 includes a first attraction member 441 provided on the first support member 32, and a second attraction member 442 provided on the second support member 34 and attractable to the first attraction member 441.

The first attraction member 441 includes a plurality of first magnets M1. The plurality of first magnets M1 is accommodated in the first accommodation portions 323 of the first support member 32. The first magnets M1 are exposed in the distal direction in the first accommodation portions 323.

The second attraction member 442 includes a plurality of second magnets M2. The plurality of second magnets M2 is accommodated in the second accommodation portions 344 of the second support member 34. The second magnets M2 are exposed in the proximal direction in the second accommodation portions 344.

As shown in FIG. 2, in an open state in which the first support member 32 and the second support member 34 move in directions approaching each other and the first and second arm portions 141 and 142 are open, the first magnets M1 and the second magnets M2 facing each other abut against each other by magnetic force. In a state in which the base portion 321 of the first support member 32 and the annular portion 341 of the second support member 34 abut against each other, relative movement of the first support member 32 and the second support member 34 in the axial direction is restricted.

The first and second attraction members 441 and 442 are not limited to being constituted by the first and second magnets M1 and M2, respectively. Only one of the first and second attraction members 441 and 442 needs to be a magnet. For example, one of the first attraction member 441 and the second attraction member 442 may be a magnet, and the other of the first attraction member 441 and the second attraction member 442 may be a metal member (made for example of a ferromagnetic material). Further, the first and second attraction members 441 and 442 are not limited to a case in which both of the first and second attraction members 441 and 442 are constituted by magnets. For example, at least one of the first and second attraction members 441 and 442 needs only be an attraction structure (such as a suction cup) capable of attracting the first support member 32 and the second support member 34 to each other in the axial direction.

As shown in FIG. 4, the distal portion of the inner rod 20 further includes a movable mechanism 46. The movable mechanism 46 tiltably supports the support structure 22 with respect to the distal portion of the inner rod 20. The movable mechanism 46 is a joint structure 461 constituted by an engaging projection 48 provided on the support structure 22 and an engaging recess 50 with which the engaging projection 48 is rotatably engaged.

The engaging projection 48 is provided at a proximal end of the first support member 32. The engaging projection 48 has a spherical shape protruding from the base portion 321 in the proximal direction. The engaging recess 50 includes a first ball support portion 521 and a second ball support portion 522 that support the engaging projection 48.

The first ball support portion 521 is provided at a distal end of the first rod 201. The first ball support portion 521 includes an opening end portion 54 that opens in the distal direction. The opening end portion 54 of the first rod 201 is deformable radially outward via a cutout portion (not shown). A part of the engaging projection 48 of the support structure 22 is inserted into the opening end portion 54. When the engaging projection 48 is inserted into the opening end portion 54, a proximal side of the engaging projection 48 is rotatably held by the opening end portion 54.

The second ball support portion 522 is provided at a distal end of the second rod 202. The second ball support portion 522 is formed in a cylindrical shape covering the first ball support portion 521 and the engaging projection 48. The second ball support portion 522 includes the engaging recess 50. The engaging recess 50 is provided inside the second ball support portion 522. An inner surface of the second ball support portion 522 is a substantially spherical surface capable of abutting against a circumferential surface of the engaging projection 48. A distal side of the engaging projection 48 is held in the engaging recess 50. The engaging projection 48 is rotatably held between the second ball support portion 522 and the opening end portion 54 of the first ball support portion 521.

That is, the support structure 22 is rotatably supported with respect to the distal end of the inner rod 20 by the movable mechanism 46 in the inner rod 20. A rotation range of the support structure 22 about an axis of the inner rod 20 is 360°.

The configuration is not limited to a configuration in which the engaging projection 48 of the movable mechanism 46 is provided on the support structure 22 and the engaging recess 50 is provided at the distal portion of the inner rod 20. For example, the distal end of the inner rod 20 may include the engaging projection 48 protruding in the distal direction, and a proximal portion of the support structure 22 may include the engaging recess 50 engageable with the engaging projection 48.

As shown in FIG. 2, the first and second arm portions 141 and 142 constitute the distal portion of the medical device 10. The first and second arm portions 141 and 142 are supported by the shaft 12 in an openable and closable manner. Each of the first and second arm portions 141 and 142 includes a pair of suction portions 58 that suction the heart 300 (see FIG. 1), a pair of rods 60 that support the suction portions 58, and an arm holder 62 that holds the rods 60. In a side view of the medical device 10 shown in FIG. 2, the first arm portion 141 and the second arm portion 142 have symmetrical shapes about an axis L of the outer rod 18 and are symmetrically disposed.

As shown in FIG. 4, the first arm portion 141 and the second arm portion 142 are disposed in an up-down direction (relative to the orientation shown in FIG. 4) about the axis L of the outer rod 18. Hereinafter, a case will be described in which the first arm portion 141 is disposed below the axis L of the outer rod 18, and the second arm portion 142 is disposed above the axis L of the outer rod 18.

As shown in FIG. 2, the suction portions 58 are respectively provided at distal ends of the first and second arm portions 141 and 142. Each suction portion 58 has a shape in which a width direction is small with respect to a longitudinal direction. The suction portions 58 are formed so as to gradually increase in width from a proximal end toward a distal end. The distal end of each suction portion 58 includes a curved portion 64, curved so as to be convex toward the distal direction. Each of the suction portions 58 has an abutment surface 66 that abuts against the surface of the heart 300 (see FIG. 1). The abutment surfaces 66 are formed flat and each includes a plurality of suction ports 68. The plurality of suction ports 68 are disposed along the longitudinal direction of the suction portion 58. Each of the plurality of suction ports 68 is connected to a suction source (not shown) via a tube 70 attached to the suction portion 58. A vacuum fluid is supplied from the suction source to each suction port 68 through the tube 70. While a plurality of suction ports 68 are illustrated, only one suction port 68 may be provided in each abutment surface 66.

The rods 60 are provided so as to extend along the longitudinal direction of the respective suction portions 58 of the first and second arm portions 141 and 142. A distal portion of each rod 60 is inserted into and connected to the respective suction portion 58. A proximal portion of each rod 60 is connected to the arm holder 62. The suction portions 58 are provided so as to be rotatable with respect to the respective rod 60 about a longitudinal axis of the rod 60.

As shown in FIG. 6, the arm holders 62 are provided at respective proximal ends of the first and second arm portions 141 and 142. The arm holder 62 of the first arm portion 141 and the arm holder 62 of the second arm portion 142 are disposed between the pair of support pieces 322 of the first support member 32 (see FIG. 4).

The proximal portion of each of the rods 60 is inserted into the respective arm holder 62. The proximal portion of each of the rods 60 is inserted so as to extend along a side surface of the respective arm holder 62. An extending direction of the rod 60, the side surface of the respective arm holder 62, and an extending direction of the respective suction portion 58 are substantially parallel. A proximal portion of each of the arm holders 62 includes a holder hole 621, and as shown in FIG. 8, the first pin member 421 of the support structure 22 is inserted through the holder hole 621. The holder hole 621 penetrates the arm holder 62 in a thickness direction of the arm holder 62. The arm holder 62 is rotatable about the first pin member 421 supported by the support pieces 322 of the first support member 32 as a fulcrum. As shown in FIG. 6, each of the arm holders 62 further includes a holder groove 622. The holder groove 622 is disposed in the distal direction with respect to the holder hole 621. The holder groove 622 penetrates the arm holder 62 in the thickness direction and is inclined from the holder hole 621 toward the side surface in the distal direction. That is, each of the holder grooves 622 is inclined with respect to the side surface of the respective arm holder 62. The second pin member 422 of the support structure 22 is inserted through the holder groove 622. As shown in FIG. 8, the second pin member 422 inserted through the holder grooves 622 of the arm holders 62 is inserted through each first pin groove 381.

In a side view of the medical device 10, the holder groove 622 of the arm holder 62 in the first arm portion 141 and the holder groove 622 of the arm holder 62 in the second arm portion 142 intersect each other. The holder grooves 622 intersect so as to be symmetrical about the first and second pin grooves 381 and 382. The first pin holes 401, the holder holes 621, and the second pin grooves 382 are disposed at the same position, and the first pin grooves 381, the holder grooves 622, and the second pin holes 402 are disposed at the same position.

As shown in FIG. 5, at a first position where the outer rod 18 has moved in the distal direction relative to the inner rod 20, the first and second arm portions 141 and 142 enter a closed state in which distal ends of the first and second arm portions 141 and 142 have rotated in a direction approaching each other about the first pin member 421 of the support structure 22 as a fulcrum.

As shown in FIG. 2, at a second position where the outer rod 18 has moved in the proximal direction relative to the inner rod 20, the first and second arm portions 141 and 142 enter an open state in which the distal ends of the first and second arm portions 141 and 142 have rotated so as to be separated from each other about the first pin member 421 of the support structure 22 as a fulcrum.

The medical device 10 further includes a rotation lock portion 72 that restricts a rotation operation of the support structure 22 with respect to the distal portion of the inner rod 20 by the movable mechanism 46. As shown in FIG. 4, the rotation lock portion 72 includes a lock member 721. The lock member 721 prevents rotation of the engaging projection 48 with respect to the engaging recess 50 by biasing the engaging recess 50 and the engaging projection 48 in directions approaching each other in the axial direction of the inner rod 20. The lock member 721 is the second ball support portion 522. The lock member 721 is movable in the axial direction by the second rod 202.

The rotation lock portion 72 includes a movement restriction portion 74 that restricts relative movement between the second rod 202 including the lock member 721 and the first rod 201. The movement restriction portion 74 includes a first threaded portion 741 provided on an inner circumferential surface of the first rod 201, and a second threaded portion 742 formed on an outer circumferential surface of the second rod 202 and screwed to the first threaded portion 741.

By relatively rotating the first rod 201 and the second rod 202, the second rod 202 is capable of being moved in the axial direction with respect to the first rod 201. The second rod 202 is moved in the proximal direction with respect to the first rod 201, and the engaging projection 48 is pushed toward the distal end of the first rod 201 by the engaging recess 50. When the engaging projection 48 is held between the lock member 721 and the distal end of the first rod 201, the rotation operation between the first rod 201 and the support structure 22 by the movable mechanism 46 is restricted, and a locked state is established in which rotation operations of the first and second arm portions 141 and 142 including the support structure 22 are prevented. The medical device 10 is not limited to a configuration including the rotation lock portion 72. For example, without providing the rotation lock portion 72, the support structure 22 and the first and second arm portions 141 and 142 may be brought into contact with the surface of the heart 300 while being kept in a rotatable state.

As seen in FIGS. 1-2, the operation portion 16 is provided at a proximal portion of the shaft 12. The operation portion 16 includes a housing 16A, a first operation member 161, and a second operation member 162. The housing 16A is formed in a tubular shape, and the proximal portion of the shaft 12 is inserted into the housing 16A. An outer surface of the housing 16A includes a slide groove 16B. The slide groove 16B is provided in a distal portion of the housing 16A. The slide groove 16B extends in an axial direction of the housing 16A. Inside the housing 16A, the inner rod 20 is fixed to a fixing portion 16C. The first operation member 161 performs opening and closing operations of the first and second arm portions 141 and 142 when operated by a user. A part of the first operation member 161 is disposed outside the housing 16A. A part of the first operation member 161 is inserted into the slide groove 16B and connected to a proximal portion of the outer rod 18 through the slide groove 16B. When the user operates the first operation member 161, the first operation member 161 moves along the slide groove 16B and moves the outer rod 18 in the axial direction relative to the inner rod 20. By moving the first operation member 161 in the axial direction relative to the inner rod 20, the outer rod 18 is movable in the axial direction.

The second operation member 162 is capable of rotating the second rod 202 relative to the first rod 201. The second operation member 162 is a rotating body rotatably provided at a proximal portion of the housing 16A. A center of the second operation member 162 is fixed to the second rod 202. The second operation member 162 and the second rod 202 are integrally rotatable. When the user operates the second operation member 162, the second rod 202 rotates relative to the first rod 201 about an axis of the second rod 202. The medical device 10 is not limited to a configuration including the operation portion 16. For example, the user may grip the outer rod 18 and manually move the outer rod 18 in the axial direction relative to the inner rod 20.

Next, a method of using the medical device 10 will be described.

As shown in FIG. 5, the medical device 10 before use (initial state) is at the first position where the outer rod 18 has moved in the distal direction relative to the inner rod 20, enters the closed state in which the distal ends of the first and second arm portions 141 and 142 have rotated in a direction approaching each other about the first pin member 421 of the support structure 22 as a fulcrum, and the proximal portions of the first and second arm portions 141 and 142 are accommodated in the arm accommodation space 26 of the outer rod 18.

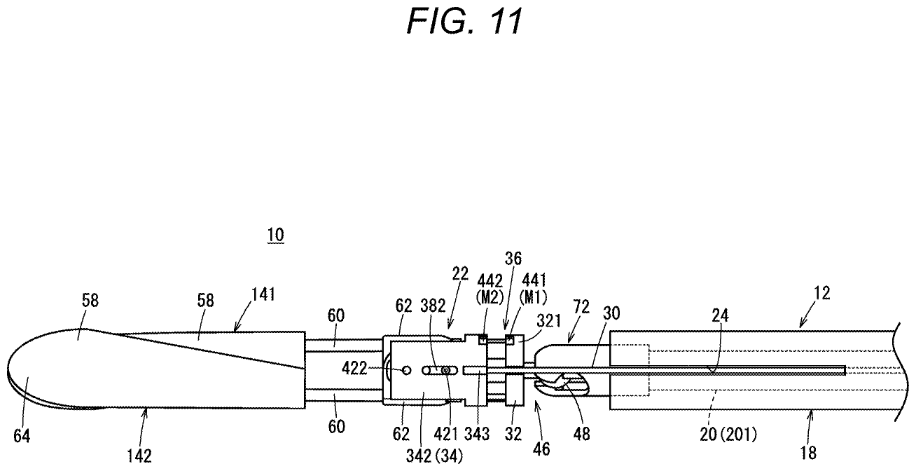

First, the distal end of the medical device 10 is inserted through the opening 304 incised adjacent to the incision 302 of the living body P shown in FIG. 1. After the distal end of the medical device 10 is inserted to a desired position relative to the heart 300 (organ) inside the living body, the user operates the first operation member 161 of the operation portion 16 and pulls the outer rod 18 in the proximal direction relative to the inner rod 20. As shown in FIG. 11, as the outer rod 18 moves in the proximal direction, the first and second arm portions 141 and 142 and the support structure 22 are exposed in the distal direction of the outer rod 18. At this time, until the proximal end of the outer rod 18 abuts against the collar member 28 (see FIG. 2), the first support member 32 and the second support member 34 are separated in the axial direction, and the closed state of the first and second arm portions 141 and 142 is maintained.

By further operation of the first operation member 161, as shown in FIG. 4, the second position is reached where the outer rod 18 has further moved in the proximal direction relative to the inner rod 20. At the second position, the proximal end of the outer rod 18 abuts against the collar member 28 and pushes the collar member 28 in the proximal direction. When the collar member 28 moves in the proximal direction together with the outer rod 18, the connecting member 30 is pulled in the proximal direction together with the collar member 28. The second support member 34 connected to the connecting member 30 moves toward the first support member 32. At this time, the first support member 32 does not move in the proximal direction.

At the second position, as shown in FIG. 8, the first and second arm portions 141 and 142 enter the open state in which the distal ends of the first and second arm portions 141 and 142 have rotated so as to be separated from each other about the first pin member 421 of the support structure 22 as a fulcrum. Specifically, when the second support member 34 moves in the proximal direction, the second pin member 422 moves in the proximal direction along the first pin grooves 381 of the support pieces 322, and the second pin member 422 moves in the proximal direction in the holder grooves 622. Accordingly, the proximal end of the arm holder 62 of the first arm portion 141 and the proximal end of the arm holder 62 of the second arm portion 142 rotate in a direction approaching each other about the first pin member 421 as a fulcrum. The distal portion of the first arm portion 141 and the distal end of the second arm portion 142 rotate in directions away from each other. The first arm portion 141 and the second arm portion 142 enter the open state in which the first arm portion 141 and the second arm portion 142 are open while being separated by a predetermined angle. In a side view of the medical device 10 shown in FIG. 2, the first arm portion 141 and the second arm portion 142 open in a V shape such that a separation distance between the first arm portion 141 and the second arm portion 142 increases toward the distal direction.

At this time, when the annular portion 341 of the second support member 34 abuts against the base portion 321 of the first support member 32, the first magnets M1 and the second magnets M2 enter a state of being in contact by magnetic force. Relative movement of the first support member 32 and the second support member 34 in the axial direction is restricted, and the open state of the first and second arm portions 141 and 142 is maintained.

After the support structure 22 is placed in a freely rotatable state with respect to the inner rod 20 by the movable mechanism 46, the abutment surfaces 66 of the suction portions 58 of the first and second arm portions 141 and 142 are brought into contact with the surface of the heart 300. When the first and second arm portions 141 and 142 abut against the surface of the heart 300, the posture of the first and second arm portions 141 and 142 changes via the movable mechanism 46 so as to conform to the surface. At this time, the support structure 22 rotates in the circumferential direction with respect to the distal portion of the inner rod 20. The surface of the heart 300 is pressed by the suction portion 58 of the first arm portion 141 and the suction portion 58 of the second arm portion 142. By rotating each suction portion 58 with respect to the rod 60, the abutment surface 66 of each suction portion 58 is capable of being brought into closer contact with the surface of the heart 300.

After the first and second arm portions 141 and 142 are brought into contact with the surface of the heart 300, the user operates the second operation member 162 of the operation portion 16, so that the rotation lock portion 72 restricts the rotation operation of the support structure 22 by the movable mechanism 46. By rotating the second rod 202 via the second operation member 162, the second rod 202 is moved in the proximal direction relative to the first rod 201 by the screwing between the first threaded portion 741 and the second threaded portion 742. The engaging projection 48 of the first support member 32 is pushed toward the distal end of the first rod 201 by the engaging recess 50. The engaging projection 48 is held between the lock member 721 and the distal end of the first rod 201. The rotation operation between the first rod 201 and the support structure 22 by the movable mechanism 46 is restricted, and a locked state is established in which rotation of the first and second arm portions 141 and 142 including the support structure 22 is prevented. That is, in a state in which each abutment surface 66 of the first and second arm portions 141 and 142 abuts against the surface of the heart 300, the rotation operations of the first and second arm portions 141 and 142 are prevented. As a result, postures of the first and second arm portions 141 and 142 with respect to the shaft 12 are fixed.

A negative pressure fluid is supplied from a suction source not shown to each suction port 68 of the first and second arm portions 141 and 142 through the tube 70. The negative pressure fluid supplied to the suction ports 68 causes the suction portions 58 of the first and second arm portions 141 and 142 to suction the surface of the heart 300. At the distal portion of the medical device 10, movement of the surface of the heart 300 caused by beating is suppressed.

After the open-chest surgery on the living body P is finished, the medical device 10 is removed from inside the living body. After supply of the negative pressure fluid to the suction portions 58 is stopped, the second operation member 162 of the operation portion 16 is rotated in a direction opposite to a direction when the rotation lock portion 72 is locked, thereby releasing the locked state of the movable mechanism 46 by the rotation lock portion 72. By rotation of the second rod 202, the engaging recess 50 is separated from the engaging projection 48, and the engaging projection 48 becomes rotatable between the engaging recess 50 and the distal end of the first rod 201.

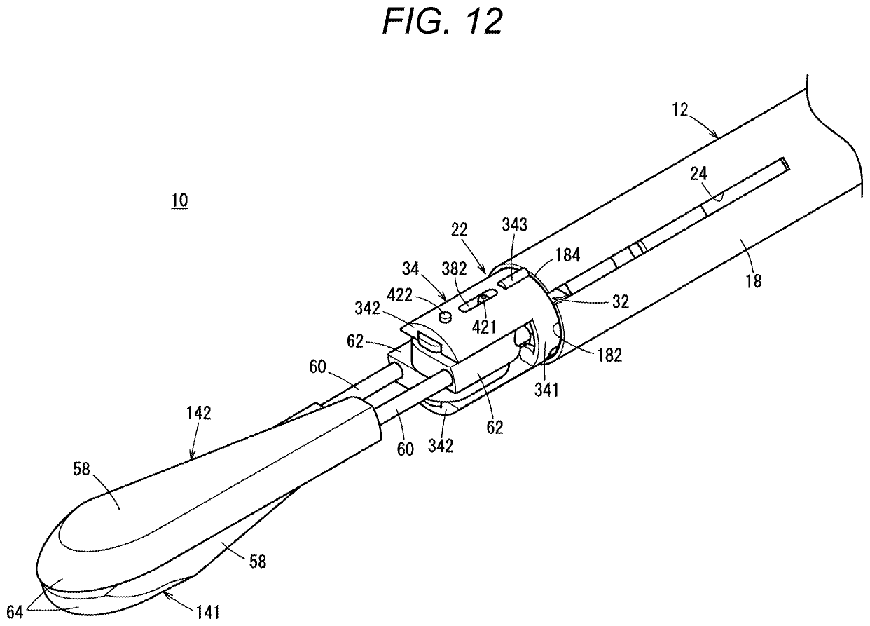

Next, the user moves the outer rod 18 in the distal direction relative to the inner rod 20 by the first operation member 161. At this time, as shown in FIG. 12, when viewed from the axial direction of the shaft 12, the slit portions 24 of the outer rod 18 and the protrusions 343 of the second support member 34 are at positions displaced in the circumferential direction of the shaft 12. Therefore, the protrusions 343 and the distal end surface 184 of the outer rod 18 face each other. This is because the first and second arm portions 141 and 142 have been rotated by the movable mechanism 46, the support structure 22 has rotated in the circumferential direction relative to the outer rod 18 together with the first and second arm portions 141 and 142, and the protrusions 343 and the slit portions 24 have displaced in the circumferential direction.

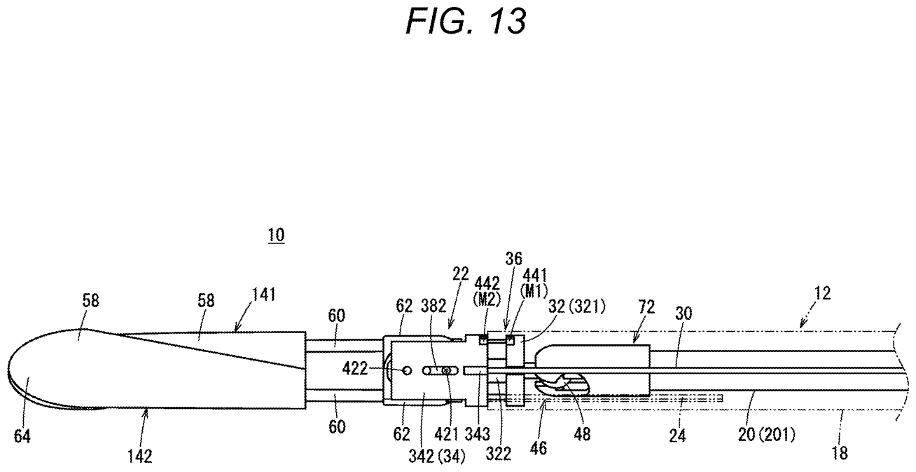

As shown in FIG. 12, the distal end of the outer rod 18 contacts the protrusions 343 of the second support member 34. By further moving the outer rod 18 in the distal direction and pushing the protrusions 343 in the distal direction by the distal end surface 184 of the outer rod 18, the second support member 34 moves relative to the first support member 32 in the distal direction. Along with the movement of the second support member 34, as shown in FIG. 13, the first magnets M1 and the second magnets M2 are separated, and a movement restriction state of the second support member 34 with respect to the first support member 32 is released. That is, in the open state of the first and second arm portions 141 and 142, the opening and closing restriction state of the first and second arm portions 141 and 142 by the arm lock portion 36 is released.

Due to the relative movement of the second support member 34 in the distal direction with respect to the first support member 32, the distal ends of the first and second arm portions 141 and 142 rotate so as to approach each other about the first pin member 421 as a fulcrum. The first and second arm portions 141 and 142 close and enter the closed state. The proximal portions of the first and second arm portions 141 and 142 are accommodated in the arm accommodation space 26 from the distal opening 182 of the outer rod 18. When the slit portions 24 of the outer rod 18 and the protrusions 343 of the second support member 34 are at positions coinciding in the circumferential direction of the shaft 12 when viewed from the axial direction of the shaft 12, the protrusions 343 move in the proximal direction along the slit portions 24, whereby the proximal portions of the first and second arm portions 141 and 142 are accommodated in the distal opening 182 of the outer rod 18, and the rods 60 of the first and second arm portions 141 and 142 are accommodated so as to close by abutting against an inner wall of the outer rod 18.

In a state in which the first and second arm portions 141 and 142 are closed, the distal portion of the medical device 10 is removed to outside the living body P through the opening 304.

The present embodiment provides the following effects.

As shown in FIG. 1, the medical device 10 includes the shaft 12 inserted into the living body P, and the openable and closable first and second arm portions 141 and 142 that include the pair of suction portions 58 for suctioning the heart 300 (organ) and are supported by the shaft 12, and the first and second arm portions 141 and 142 constitute the distal portion of the medical device 10.

Accordingly, since the first and second arm portions 141 and 142 are openable and closable, the first and second arm portions 141 and 142 are capable of being inserted into the living body through the opening 304 provided in the living body P in a state in which the first and second arm portions 141 and 142 are closed. Therefore, unlike a structure in which a cap is removed from a distal end of a shaft inside a living body and a stabilizer is attached to the distal end of the shaft, occurrence of an event in which the cap is dropped into a surgical field is avoided.

As shown in FIG. 2, the shaft 12 includes the outer rod 18 having a tubular shape, the inner rod 20 inserted through the lumen 181 of the outer rod 18, and the support structure 22 provided at the distal end of the inner rod 20. The support structure 22 supports the first and second arm portions 141 and 142 in an openable and closable manner, and as shown in FIG. 5, the outer rod 18 includes the arm accommodation space 26 capable of accommodating the proximal portions of the first and second arm portions 141 and 142 in a state in which the first and second arm portions 141 and 142 are closed.

Accordingly, since the first and second arm portions 141 and 142 are accommodated in the arm accommodation space 26 of the outer rod 18 in a state in which the first and second arm portions 141 and 142 are closed, insertion resistance of the first and second arm portions 141 and 142 is effectively suppressed when the first and second arm portions 141 and 142 are inserted into the living body.

As shown in FIG. 5, at the first position where the outer rod 18 has moved in the distal direction relative to the inner rod 20, the first and second arm portions 141 and 142 are placed in the closed state in which the distal ends of the first and second arm portions 141 and 142 have rotated in a direction approaching each other about a support portion (the first pin member 421) of the support structure 22 as a fulcrum, and as shown in FIG. 2, at the second position where the outer rod 18 has moved in the proximal direction relative to the inner rod 20, the first and second arm portions 141 and 142 are placed in the open state in which the distal ends of the first and second arm portions 141 and 142 have rotated so as to be separated from each other.

Accordingly, the first and second arm portions 141 and 142 are inserted into the living body at the first position where the first and second arm portions 141 and 142 are in the closed state, and the first and second arm portions 141 and 142 are rotated inside the living body to be set to the opened second position, so that both insertability of the first and second arm portions 141 and 142 into the living body and retainability of the surface of the heart 300 are capable of being achieved.

As shown in FIG. 2, the medical device 10 includes the operation portion 16 (first operation member 161) that is provided at the proximal portion of the outer rod 18 and performs opening and closing operations of the first and second arm portions 141 and 142. Accordingly, by operating the operation portion 16, the opening and closing operations of the first and second arm portions 141 and 142 are capable of being easily performed.

The operation portion 16 moves the outer rod 18 in the axial direction relative to the inner rod 20. Accordingly, by operating the operation portion 16, the outer rod 18 is capable of being easily moved in the axial direction relative to the inner rod 20.

As shown in FIG. 2, in the open state of the first and second arm portions 141 and 142, when the outer rod 18 is moved in the distal direction relative to the inner rod 20 from the second position as shown in FIG. 11, the first and second arm portions 141 and 142 enter the closed state in which the distal ends of the first and second arm portions 141 and 142 have rotated in a direction approaching each other, and therefore, when the first and second arm portions 141 and 142 are removed from inside the living body after surgery, the first and second arm portions 141 and 142 are capable of being closed and taken out in a compact state.

As shown in FIG. 2, the outer rod 18 includes the slit portion 24 that opens in the distal end surface 184 of the outer rod 18 and extends from the distal end surface 184 in the proximal direction. As shown in FIG. 9, in a state in which the proximal portions of the first and second arm portions 141 and 142 are accommodated in the arm accommodation space 26 of the outer rod 18, the protrusions 343 provided on the support structure 22 are inserted into the slit portions 24. When the first and second arm portions 141 and 142 are placed in the open state, the protrusions 343 of the support structure 22 are disengaged from the slit portions 24.

Accordingly, when the outer rod 18 and the inner rod 20 are relatively moved in the axial direction by operation of the operation portion 16, the support structure 22 is capable of being guided in the axial direction of the outer rod 18 by the slit portions 24.

As shown in FIG. 6, the support structure 22 includes the first support member 32 provided at the distal portion of the inner rod 20, the second support member 34 provided so as to be relatively movable in the axial direction with respect to the first support member 32, and the protrusions 343 provided on an outer wall of the second support member 34 and protruding radially outward. After the entirety of the first and second arm portions 141 and 142 protrudes from the distal opening 182 of the outer rod 18 and enters the open state, when the outer rod 18 is moved in the distal direction relative to the inner rod 20 in a state in which the protrusions 343 and the distal end surface 184 of the outer rod 18 face each other in the axial direction, the protrusions 343 abut against the distal end of the outer rod 18, and the second support member 34 moves relative to the first support member 32 in the distal direction, so that the first and second arm portions 141 and 142 are capable of being placed in the closed state.

As shown in FIG. 2, the support structure 22 includes the arm lock portion 36 that releasably restricts opening and closing operations of the first and second arm portions 141 and 142 in the open state of the first and second arm portions 141 and 142. The arm lock portion 36 includes the first attraction member 441 provided on the first support member 32, and the second attraction member 442 provided on the second support member 34 and attractable to the first attraction member 441.

Accordingly, in the open state of the first and second arm portions 141 and 142, the first attraction member 441 and the second attraction member 442 are attracted to each other, and relative movement of the first support member 32 and the second support member 34 in the axial direction is restricted, so that the open state of the first and second arm portions 141 and 142 is effectively maintained.

As shown in FIG. 12, when the outer rod 18 moves in the distal direction relative to the inner rod 20 from the second position, which is the open state of the first and second arm portions 141 and 142, in a state in which the protrusions 343 and the distal end surface 184 of the outer rod 18 face each other in the axial direction, the protrusions 343 abut against the distal end surface 184 of the outer rod 18 and push the protrusions 343 in the distal direction. Along with movement of the outer rod 18, an attraction state between the first attraction member 441 and the second attraction member 442 is released.

Accordingly, when the first and second arm portions 141 and 142 are accommodated in the outer rod 18 after surgery, the distal end of the outer rod 18 is brought into contact with the protrusions 343, so that the open state of the first and second arm portions 141 and 142 by the arm lock portion 36 is capable of being released and the first and second arm portions 141 and 142 are capable of being closed. By recognizing that the protrusions 343 have abutted against the distal end of the outer rod 18 without directly visually confirming the abutment, the user is capable of effectively confirming that the first and second arm portions 141 and 142 have entered a state of being accommodated in the arm accommodation space 26 of the outer rod 18.

As shown in FIG. 6, the first attraction member 441 and the second attraction member 442 are the first and second magnets M1 and M2, respectively. Accordingly, by forming the first and second attraction members 441 and 442 from the first and second magnets M1 and M2, respectively, relative movement of the first support member 32 and the second support member 34 is capable of being effectively restricted.

As shown in FIG. 4, the medical device 10 includes the movable mechanism 46 that is provided at the distal portion of the inner rod 20 and rotatably supports the support structure 22 with respect to the distal portion of the inner rod 20. The movable mechanism 46 is the joint structure 461 including the engaging projection 48 having a spherical shape provided on the support structure 22, and the engaging recess 50 provided in the inner rod 20 and with which the engaging projection 48 is rotatably engaged.

Accordingly, by rotating the support structure 22 at the distal portion of the inner rod 20 by the movable mechanism 46, the first and second arm portions 141 and 142 are capable of being effectively brought into contact with a desired contact site on the heart 300.

The medical device 10 includes the rotation lock portion 72 that restricts a rotation operation of the support structure 22 with respect to the inner rod 20 by the movable mechanism 46. The rotation lock portion 72 includes the lock member 721 that prevents rotation of the engaging projection 48 with respect to the engaging recess 50 by biasing the engaging recess 50 and the engaging projection 48 in directions approaching each other.

Accordingly, in a state in which the first and second arm portions 141 and 142 are rotated with respect to the distal portion of the inner rod 20 by the movable mechanism 46 and brought into contact with the contact site of the heart 300, the rotation operation of the support structure 22 is locked by the lock member 721 of the rotation lock portion 72, so that a contact state of the first and second arm portions 141 and 142 with respect to the surface of the heart 300 is effectively maintained.

As shown in FIG. 3, the distal ends of the first and second arm portions 141 and 142 include the curved portions 64 curved so as to be convex toward the distal ends. Accordingly, when the first and second arm portions 141 and 142 are inserted into the living body, insertion resistance and a burden on the living body P are capable of being reduced.

The present invention is not limited to the above-described disclosure, and various configurations may be adopted without departing from the gist of the present invention.

What is claimed is:

1. A medical device that suppresses movement of a surface of an organ of a living body by being brought into contact with the organ, the medical device comprising:
a shaft that is inserted into the living body; and
a pair of arm portions that constitute a distal portion of the medical device, include a pair of suction portions that suction the organ, and are supported by the shaft, the pair of arm portions being openable and closable.

2. The medical device according to claim 1, wherein
the shaft includes
an outer rod having a tubular shape,
an inner rod inserted through a lumen of the outer rod, and
a support structure provided at a distal end of the inner rod and supporting the pair of arm portions in an openable and closable manner,
the outer rod and the inner rod are relatively movable in an axial direction of the outer rod, and
the outer rod includes an arm accommodation space capable of accommodating at least proximal portions of the pair of arm portions in a state in which the pair of arm portions is closed.

3. The medical device according to claim 2, wherein
at a first position where the outer rod has moved in a distal direction relative to the inner rod, the pair of arm portions enter a closed state in which distal ends of the pair of arm portions have rotated in a direction approaching each other about a support portion of the support structure as a fulcrum, and
at a second position where the outer rod has moved in a proximal direction relative to the inner rod, the pair of arm portions enter an open state in which the distal ends of the pair of arm portions have rotated so as to be separated from each other about the support portion of the support structure as a fulcrum.

4. The medical device according to claim 3, comprising:
an operation portion that is provided at a proximal portion of the outer rod and performs opening and closing operations of the pair of arm portions.

5. The medical device according to claim 4, wherein
the operation portion moves the outer rod in the axial direction relative to the inner rod.

6. The medical device according to claim 3, wherein
in the open state of the pair of arm portions, when the outer rod is moved from the second position in the distal direction relative to the inner rod, the pair of arm portions enter the closed state in which the distal ends of the pair of arm portions have rotated in a direction approaching each other about the support portion of the support structure as a fulcrum.

7. The medical device according to claim 3, wherein
the outer rod includes a slit portion that opens in a distal end surface of the outer rod and extends from the distal end surface in the proximal direction,
in a state in which at least the proximal portions of the pair of arm portions are accommodated in the arm accommodation space of the outer rod, a protrusion provided on the support structure is inserted into the slit portion, and
in a state in which the pair of arm portions are placed in the open state by moving the outer rod in the proximal direction relative to the inner rod, the protrusion of the support structure is disengaged from the slit portion.

8. The medical device according to claim 7, wherein
the support structure includes
a first support member provided at a distal portion of the inner rod,
a second support member provided so as to be relatively movable in the axial direction with respect to the first support member, and
the protrusion provided on an outer wall of the second support member and protruding radially outward, and
after an entirety of the pair of arm portions protrudes from a distal opening of the outer rod and the pair of arm portions enters the open state, when the outer rod is moved in the distal direction relative to the inner rod in a state in which the protrusion and the distal end surface of the outer rod face each other in the axial direction, the protrusion abuts against a distal end of the outer rod, and the second support member moves relative to the first support member in the distal direction, so that the pair of arm portions enters the closed state.

9. The medical device according to claim 2, wherein
the support structure includes
a first support member provided at a distal portion of the inner rod,
a second support member provided so as to be relatively movable in the axial direction with respect to the first support member,
a protrusion provided on an outer wall of the second support member and protruding radially outward, and
an arm lock portion that releasably restricts opening and closing operations of the pair of arm portions in an open state of the pair of arm portions,
the arm lock portion includes
a first attraction member provided on the first support member, and
a second attraction member provided on the second support member and attractable to the first attraction member, and
in the open state of the pair of arm portions, the first attraction member and the second attraction member are attracted to each other, and relative movement of the first support member and the second support member in the axial direction is restricted.

10. The medical device according to claim 9, wherein
the outer rod includes a slit portion that opens in a distal end surface of the outer rod and extends from the distal end surface in a proximal direction,
at a first position that is a closed state of the pair of arm portions, the protrusion is inserted through the slit portion of the outer rod,
at a second position that is the open state of the pair of arm portions, the protrusion is disengaged from the slit portion and disposed on a distal side with respect to the distal end surface of the outer rod, and
when the outer rod moves in a distal direction relative to the inner rod from the second position in a state in which the protrusion and the distal end surface of the outer rod face each other in the axial direction, the protrusion abuts against the distal end surface of the outer rod and pushes the protrusion in the distal direction, so that an attraction state between the first attraction member and the second attraction member is released.

11. The medical device according to claim 9, wherein
at least either one of the first attraction member or the second attraction member is a magnet.

12. The medical device according to claim 2, comprising:
a movable mechanism that is provided at a distal portion of the inner rod and tiltably supports the support structure with respect to the distal portion of the inner rod,
the movable mechanism being a joint structure including
an engaging projection having a spherical shape provided on either one of the support structure or the inner rod, and
an engaging recess provided on another of the support structure or the inner rod and with which the engaging projection is rotatably engaged.

13. The medical device according to claim 12, comprising:
a rotation lock portion that restricts a rotation operation of the support structure with respect to the inner rod by the movable mechanism, wherein
the rotation lock portion includes a lock member that prevents rotation of the engaging projection with respect to the engaging recess by biasing the engaging recess and the engaging projection in directions approaching each other.

14. The medical device according to claim 1, wherein
a distal end of each arm portion of the pair of arm portions includes a curved portion curved so as to be convex toward the distal end.

15. A medical device that suppresses movement of a surface of an organ of a living body by being brought into contact with the organ, the medical device comprising:
a shaft that is inserted into the living body; and
a pair of arm portions that constitute a distal portion of the medical device, include a pair of suction portions that suction the organ, and are supported by the shaft, the pair of arm portions being openable and closable; and
wherein the shaft includes:
an outer rod having a tubular shape,
an inner rod inserted through a lumen of the outer rod, and
a support structure provided at a distal end of the inner rod and supporting the pair of arm portions in an openable and closable manner,
the outer rod and the inner rod are relatively movable in an axial direction of the outer rod, and
the outer rod includes an arm accommodation space capable of accommodating at least proximal portions of the pair of arm portions in a state in which the pair of arm portions is closed; and
wherein a distal end of each arm portion of the pair of arm portions includes a curved portion curved so as to be convex toward the distal end.

16. The medical device according to claim 15, wherein each of the suction portions includes at least one suction port configured to suction the organ.

17. A medical device that suppresses movement of a surface of an organ of a living body by being brought into contact with the organ, the medical device comprising:
a shaft that is inserted into the living body; and
a pair of arm portions that constitute a distal portion of the medical device, include a pair of suction portions that suction the organ, and are supported by the shaft, the pair of arm portions being openable and closable by pivoting about a fulcrum.

18. The medical device according to claim 17, wherein
the shaft includes
an outer rod having a tubular shape,
an inner rod inserted through a lumen of the outer rod, and
a support structure provided at a distal end of the inner rod and supporting the pair of arm portions in an openable and closable manner,
the outer rod and the inner rod are relatively movable in an axial direction of the outer rod, and
the outer rod includes an arm accommodation space capable of accommodating at least proximal portions of the pair of arm portions in a state in which the pair of arm portions is closed.

19. The medical device according to claim 18, comprising:
an operation portion that is provided at a proximal portion of the outer rod and performs opening and closing operations of the pair of arm portions.
